(19) United States
(12) Patent Application Publication
(10) Pub. No.: US 2026/0051398 A1
(43) Pub. Date: Feb. 19, 2026

(54) SYSTEM AND METHODS FOR SURGICAL COLLABORATION

(71) Applicant: Kaliber Labs Inc., San Francisco, CA (US)

(72) Inventors: Chandra JONELAGADDA, San Francisco, CA (US); Rithesh PUNYAMURTHULA, San Francisco, CA (US); Richard ANGELO, San Francisco, CA (US)

(21) Appl. No.: 19/101,587

(22) PCT Filed: Aug. 7, 2023
(86) PCT No.: PCT/US2023/029673

§ 371 Date: Feb. 5, 2025

Related U.S. Application Data
(60) Provisional application No. 63/395,770, filed on Aug. 5, 2022.

(51) Int. Cl. G16H 40/67; A61B 5/055; A61B 34/10; A61B 90/00; G06T 7/00; G06T 7/73; G06T 19/00; G06T 19/20; G16H 20/40

(52) U.S. Cl. CPC G16H 40/67; A61B 5/055; A61B 34/10; A61B 90/37; G06T 7/0014; G06T 7/75; G06T 19/006; G06T 19/20; G16H 20/40; A61B 2034/105; G06T 2207/30008; G06T 2219/2016

(57) ABSTRACT

Apparatuses, systems, and methods are disclosed to manage and process surgical data, and enable collaboration between two or more surgeons or other clinicians. A repository may be created that includes surgical data (including video data and/or 2024/030683 or radiological image data) that may be selectively shared between medical professionals. One or more trained neural networks may process the annotated surgical reports in order to populate the repository. Additional trained neural networks may generate surgical recommendations in response to user requests, based on contents of the repository. Other trained neural networks may generate surgical templates to guide a surgeon during an operation.

### CLAIM OF PRIORITY

This application claims priority to U.S. Provisional Ser. No. 63/395,770, , filed Aug. 5, 2022, and titled “SYSTEM AND METHODS FOR SURGICAL COLLABORATION,” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present disclosure relates generally to surgery and more specifically to enabling cloud-based surgical collaboration between surgeons and/or other medical professionals.

### BACKGROUND

Surgeons often consult their peers when faced, or anticipate being faced, with complex situations. Surgeons may also share the details of complex cases which they handled, to receive or elicit feedback and validation from their peers. The value of this interaction between surgeons is evidenced by the fact that most surgeon/surgery focused events may have several panel discussions where cases are presented by surgeons or panel of surgeons for analysis and feedback from their peers.

However, some surgeons who participate in these discussions often walk away with only a partial knowledge of the case, the patient's medical history, and the nuances involved in treating the patient. Secondly, it is also difficult and time-consuming for the surgeons to prepare materials for these presentations.

### SUMMARY OF THE DISCLOSURE

Described herein are apparatuses, systems, and methods to manage and process surgical data, and enable collaboration between two or more surgeons or other clinicians. A repository may be created that includes surgical data (including video data and/or radiological image data). Contents of the repository may be selectively shared between medical professionals enabling the sharing of surgical knowledge and procedures.

Any of the methods described herein may be used to upload and create a plurality of annotated surgical reports. The annotated surgical reports may be collected to form the repository. The repository may include video highlights, textual and audio annotations and the like that may form a surgical recommendation for one or more surgical procedures. In some examples, a trained neural network may process the annotated surgical reports to populate the repository. Additional trained neural networks may generate surgical recommendations in response to user requests. In some examples, trained neural networks may generate surgical templates to guide a surgeon during an operation.

Any of the methods described herein may be used to create an annotated surgical report. Any of the methods described herein may include obtaining surgical data, annotating, via a processor, the surgical data generating annotated surgical data, and generating an annotated surgical report based at least in part on the annotated surgical data.

In any of the methods described herein, the surgical data may include surgical video data, radiological image data, or a combination thereof. In any of the methods described herein, the annotated surgical report may be stored in a cloud-based storage device. In some examples, the surgical data may be redacted to remove patient identifying data.

In any of the methods described herein, annotating the surgical data may include determining a start and end time of the surgical data. Any of the methods described herein may further include receiving, from a surgeon, annotation information associated with the surgical data. Furthermore, the annotation information may include at least one of voice annotation, text annotation, and overlay annotation associated with the surgical data.

Any of the systems described herein may include one or more processors and a memory configured to store instructions that, when executed by one of the one or more processors, cause the system to obtain surgical data, annotate the surgical data generating annotated surgical data, and generate an annotated surgical report based at least in part on the annotated surgical data.

Any of the non-transitory computer-readable storage mediums described herein may include instructions that, when executed by one or more processors of a device, cause the device to perform operations comprising obtaining surgical data, annotating, via a processor, the surgical data generating annotated surgical data, and generating an annotated surgical report based at least in part on the annotated surgical data.

Any of the methods described herein may create a repository of collaboration data. The methods may include receiving one or more annotated surgery reports, and generating a repository of collaboration data based on the one or more annotated surgery reports, wherein the repository of collaboration data includes metadata, surgical highlights, and a technique repository.

In any of the methods described herein, generating the repository of collaboration data may include executing a neural network trained to recognize an anatomical area within any of the annotated surgery reports, and the recognized anatomical area may be added to at least one of the metadata, the surgical highlights, and the technique repository.

In any of the methods described herein, generating a repository of collaboration data may include executing a neural network trained to recognize a surgical area within any of the annotated surgery reports, and the recognized surgical area is added to at least one of the metadata, the surgical highlights, and the technique repository.

In any of the methods described herein, the annotated surgery reports may include video image data, wherein identifying patient information has been removed from the video image data. Furthermore, in any of the methods described herein generating the repository of collaboration data may include executing a neural network trained to recognize whether a surgical procedure has begun or is proceeding.

In any of the methods described herein, generating the repository of collaboration data may include executing a neural network trained to recognize at least one of surgical tools and surgical implants, and the at least one of surgical tools and surgical implants is added to at least one of the metadata, the surgical highlights, and the technique repository.

In any of the methods described herein, generating the repository of collaboration data may include executing a neural network trained to recognize at least one of sutures and anchors within a surgical area, and the at least one of recognized sutures and anchors is added to at least one of the metadata, the surgical highlights, and the technique repository.

In any of the methods described herein, generating the repository of collaboration data may include executing a neural network trained to recognize when at least one of a surgical tool or surgical implant is used for a surgical procedure, and the surgical procedure is added to at least one of the metadata, the surgical highlights, and the technique repository.

In any of the methods described herein, generating the repository of collaboration data may include executing a neural network trained to recognize scene changes within image data, wherein the recognized scene changes are added to at least one of the metadata, the surgical highlights, and the technique repository.

Any of the systems described herein may include one or more processors and a memory configured to store instructions that, when executed by one of the one or more processors, cause the system to receive one or more annotated surgery reports and generate a repository of collaboration data based on the one or more annotated surgery reports, wherein the repository of collaboration data includes metadata, surgical highlights, and a technique repository.

Any of the non-transitory computer-readable storage mediums described herein may include instructions that, when executed by one or more processors of a device, cause the device to perform operations comprising receiving one or more annotated surgery reports and generating a repository of collaboration data based on the one or more annotated surgery reports, wherein the repository of collaboration data includes metadata, surgical highlights, and a technique repository.

Any of the methods described herein may provide surgical guidance. The methods may include receiving a request for surgical guidance, executing, by a processor, a neural network trained to match terms within the request for surgical guidance with metadata associated with surgery reports, and providing surgery reports that include metadata which match terms within the request for surgical guidance.

In any of the methods described herein, the neural network may be trained based on interactions between two or more surgeons regarding a similar surgical subject matter.

Furthermore, in any of the methods described herein, the neural network may be based at least in part on a surgical area within the request for surgical guidance.

In any of the methods described herein, the metadata may include patient information, radiological findings, clinical notes, or a combination thereof. In any of the methods described herein, the provided surgery reports may include a surgical highlight video.

Any of the systems described herein may include one or more processors, and a memory configured to store instructions that, when executed by one of the one or more processors, cause the system to receive a request for surgical guidance, execute, by a processor, a neural network trained to match terms within the request for surgical guidance with metadata associated with surgery reports, and provide surgery reports that include metadata which match terms within the request for surgical guidance.

Any of the non-transitory computer-readable storage medium described herein may include instructions that, when executed by one or more processors of a device, cause the device to perform operations comprising receiving a request for surgical guidance, executing, by a processor, a neural network trained to match terms within the request for surgical guidance with metadata associated with surgery reports, and providing surgery reports that include metadata which match terms within the request for surgical guidance.

Any of the methods described herein may create a surgical template of an operating surgeon. The method may include receiving a request for a surgical template, executing, by a processor, a neural network trained to match terms within the request for the surgical template with metadata associated with at least one highlight video, providing a surgical template that includes metadata which match terms within the request for a surgical template, wherein the surgical template includes the highlight video.

In any of the methods described herein, the neural network may be trained based on a weighted recommendation of surgical peers and an expert cohort. In any of the methods described herein, the highlight video may be overlayed over a real-time surgery video feed. Furthermore, the highlight video may be deactivated after review by the operating surgeon.

In any of the methods described herein, the surgical template may include locations for anchors for a surgical repair. In any of the methods described herein, the surgical template may include a location for anchors based on bone loss. In any of the methods described herein, the surgical template may include a location for a tunnel placement in conjunction with anterior cruciate ligament (ACL) reconstruction surgeries.

Any of the systems described herein may include one or more processors and a memory configured to store instructions that, when executed by one of the one or more processors, cause the system to receive a request for a surgical template, execute, by a processor, a neural network trained to match terms within the request for the surgical template with metadata associated with at least one highlight video, and provide a surgical template that includes metadata which match terms within the request for a surgical template, wherein the surgical template includes the highlight video.

Any of the non-transitory computer-readable storage mediums described herein may include instructions that, when executed by one or more processors of a device, cause the device to perform operations comprising receiving a request for a surgical template, executing, by a processor, a neural network trained to match terms within the request for the surgical template with metadata associated with at least one highlight video, and providing a surgical template that includes metadata which match terms within the request for a surgical template, wherein the surgical template includes the highlight video.

Any of the methods described herein may generate a surgical template. The methods may include receiving one or more still images associated with a surgical procedure, receiving one or more radiological images, determining location offsets of one or more implant anchors based on the one or more still images and the one or more radiological images, and displaying, on a video display, the determined location offsets.

In any of the methods described herein, the one or more still images may be from a video feed of an ongoing surgery. In some examples, the determined location offsets may be overlayed over a live video feed of an ongoing surgery.

In any of the methods described herein, determining the location offsets may include analyzing, by a processor executing a trained neural network, anatomical differences between the one or more still images and the one or more radiological images.

In some examples, any of the methods may include determining a relative position of at least one of a tool or implant with respect to an anatomical structure. In still other examples, any of the methods described herein may include recognizing, by a processor executing a trained neural network, a pathology in the one or more still images.

In any of the methods described herein, determining the location offsets may be performed when a field of view of the one or more still images match at least a portion of the one or more radiological images.

In any of the methods described herein, the radiological images may include x-ray images, magnetic resonance images (MRI), or a combination thereof. In some examples, any of the methods described herein may include determining an approach angle of a drill in response to determining the location offsets. In any of the methods described herein may include receiving, from a surgeon, a confirmation that at least one radiological image matches at least one still image.

Any of the non-transitory computer-readable storage mediums described herein may include instructions that, when executed by one or more processors of a device, cause the device to perform operations comprising receiving one or more still images associated with a surgical procedure, receiving one or more radiological images, determining location offsets of one or more implant anchors based on the one or more still images and the one or more radiological images, and displaying, on a video display, the determined location offsets.

Any of the non-transitory computer-readable storage mediums described herein may further include instructions for overlaying the determined location offsets over a live view feed of an ongoing surgery. In some examples, the non-transitory computer-readable storage mediums'instructions for determining the location offsets include instructions for analyzing anatomical differences between the one or more still images and the one or more radiological images.

Any of the non-transitory computer-readable storage mediums described herein may further comprise instructions for determining a relative position of at least one of a tool or implant with respect to an anatomical structure. In some examples, the non-transitory computer-readable storage medium may further include instructions for recognizing, by a processor executing a trained neural network, a pathology in the one or more still images.

In general, instructions for determining the location offsets may be when a field of view of the one or more still images match at least a portion of one or more radiological images. In many aspects, the radiological images may include x-ray images, magnetic resonance images (MRI), or a combination thereof.

Any of the non-transitory computer-readable storage mediums described herein may further comprise instructions for determining an approach angle of a drill in response to determining the location offsets. Any of the non-transitory computer-readable storage mediums described herein may further comprise instructions for receiving, from a surgeon, a confirmation that at least one radiological image matches at least one still image.

Any of the systems described here may include one or more processors and a memory configured to store instructions that, when executed by one of the one or more processors, cause the system to receive one or more still images associated with a surgical procedure, receive one or more radiological images, determine location offsets of one or more implant anchors based on the one or more still images and the one or more radiological images, and display, on a video display, the determined location offsets.

As mentioned, described herein are methods and apparatuses (e.g., systems and devices, including software) for assisting a user (e.g., surgeon, doctor, physician, nurse, etc.) in performing a medical procedure using one or more templates. Any of these surgical templates may be used in real time. During the surgery, the surgical template may be imported into the surgical assistance system (e.g., including surgical assistance software) and may be used to provide guidance to the surgeon. These apparatuses may analyze a received video feed in real time, match the images in the field of view (FOV) to segmented images from a patient's pre-surgical scan (e.g., MRI scan(s)). This matched data (e.g., matched pair) may be used to ascribe physical dimensions to the images in the FOV. In some examples the methods may include generating a 3D model from the pre-scan data and/or the real-time images.

For example, the template for a particular medical (e.g., surgical) procedure may be imported and the user (e.g., surgeon) may be alerted when the real-time surgical field of view matches a view specified in the template. Once the user confirms that the view has been matched, the template can be activated, and the 3D model (e.g., obtained by matching the field of view and the patient's MRI and the corresponding images from the template) may be update and/or displayed; the 3D model may reflect the surgical field of view and may be updated with the template.

For example, matching may provide a mapping between the recommendations in the template and field of view. Various instructions, such as offsets, measurements, zones to avoid, etc., may be mapped from the template to the field of view along corresponding anatomical structures. The overlays may be visualized in the field of view, where they may be used by the surgeon to perform the procedure. The surgeon can choose to follow or ignore the recommendations in the template.

The system (which may include a real-time processing engine, as described herein) may match the views stipulated in the templates to the views achieved in the field of view and may provide visual confirmation that the surgeon achieves the intermediate views at critical stages in the surgery. For example, the apparatus (e.g., using a view recognition engine) may alert the surgeon that he/she is not properly positioned to deliver a given implant. Improper positioning could result in the delivery of anchors at incorrect angles. In other cases, failures to achieve the proper view could result in failures to achieve proper implant positioning. Other cues could also be provided to aid the surgeon, and any of these methods and apparatuses may use view classification to analyzes the pathology and anatomical structures in the field of view and may produce a different kind of alert, e.g., indicating that the target site might not have been prepared to the specifications in the template. The user could again choose to ignore the alert after determining that this site is appropriate for the patient.

For example, an algorithm used to match the field of view and the template may use the pre-surgical scan(s) of the patient (e.g., radiological images such as, but not limited to MRI scans). The physical dimensions of the anatomical structures seen in the field of view may be scaled to the patient's pre-surgical scan(s). The offsets and layout in the template may be specified in relation to major, procedure specific, anatomical structures seen in the pre-surgical scan(s). The relative offsets may be mapped to physical dimensions by matching the corresponding reference anatomical structure in the patient's MRI and obtaining its dimension.

Once the physical dimensions are computed, the MRI-FOV matching algorithms estimates the offsets and implants positions by matching the AI-generated, segmented, masks of corresponding anatomical structures.

For example, described herein are systems for assisting in a surgical procedure. A system may include: one or more processors; and optionally one or more displays, and a memory coupled to the one or more processors, the memory storing computer-program instructions, that, when executed by the one or more processors, perform a computer-implemented method comprising: receiving a real-time video feed of a surgical field of view; matching a field of view specified in a surgical procedure template to the surgical field of view to generate a 3D model of the surgical field of view; activating the surgical procedure template after a user confirms that the field of view of the surgical procedure template matches the surgical field of view; transferring overlays from the activated template to the 3D model, wherein the overlays comprise one or more of: offsets, measurements, or zones to avoid; displaying the 3D model with the overlays to the user; updating, in real time, the displayed 3D model with the overlays as the surgical field of view changes from the received real-time video feed; extracting procedure data from the real-time video feed in real time; identifying, by the one or more processors in real time, a match between a predefined procedural landmark from the surgical procedure template and the extracted procedure data; and displaying visual confirmation on the display that user has matched the predefined procedural landmark.

The computer-implemented method may further comprise: matching one or more pre-surgical patient scans to the 3D model. The one or more pre-surgical patient scan(s) may comprise one or more MRI scans or other radiological scan(s).

Updating the displayed 3D model with the overlays as the surgical field of view changes from the received real-time video feed may further comprise updating the overlays. The procedure data may comprise the surgical field of view or a modified version of the field of view. In some examples the procedure data comprises one or more of: visual data of implant position, surgical tool position, or anatomy orientation.

Any of these systems and/or methods may include identifying, by the one or more processors in real time, a mismatch between the predefined procedural landmark from the surgical procedure template and the extracted procedure data. The system and/or method may include displaying visual confirmation on the display that user has not matched the predefined procedural landmark. For example, any of these systems and/or methods may include: scaling physical dimensions in the 3D model using a pre-surgical scan for the patient, and/or adjusting the template based on one or more structures from pre-surgical scan for the patient. For example, adjusting the template may comprise adjusting based on a physical dimension from a corresponding reference anatomical structure from a matched pre-surgical scan for the patient. Adjusting the template may comprise one or more of: scaling, referencing, labeling, or measuring. In some examples adjusting the template comprises adjusting one or more of: the offsets or layout in the template. The one or more structures may comprise an anatomical structure or a procedure-specific structure.

Any of these systems and/or methods may be configured to import the surgical procedure template.

For example, a system for assisting in a surgical procedure may include: a memory coupled to the one or more processors, the memory storing computer-program instructions, that, when executed by the one or more processors, perform a computer-implemented method comprising: receiving a real-time video feed of a surgical field of view; matching a field of view specified in a surgical procedure template to the surgical field of view to generate a 3D model of the surgical field of view; activating the surgical procedure template after a user confirms that the field of view of the surgical procedure template matches the surgical field of view; transferring overlays from the activated template to the 3D model, wherein the overlays comprise one or more of: offsets, measurements, or zones to avoid; displaying the 3D model with the overlays to the user; updating, in real time, the displayed 3D model with the overlays as the surgical field of view changes from the received real-time video feed; extracting, in real time, procedure data from the real-time video feed, wherein the procedure data comprises the surgical field of view or a modified version of the field of view; identifying, by the one or more processors in real time, a mismatch between the predefined procedural landmark from the surgical procedure template and the extracted procedure data; displaying visual confirmation on the display that user has not matched the predefined procedural landmark. As mentioned, any of these systems may include one or more displays (e.g., screens, etc.).

Also described herein is the software, e.g., the non-transitory computer-readable storage medium comprising instructions that, when executed by one or more processors of a device, cause the device to perform the operations including any of the operations described above, such as: receiving a real-time video feed of a surgical field of view; matching a field of view specified in a surgical procedure template to the surgical field of view to generate a 3D model of the surgical field of view; activating the surgical procedure template after a user confirms that the field of view of the surgical procedure template matches the surgical field of view; transferring overlays from the activated template to the 3D model, wherein the overlays comprise one or more of: offsets, measurements, or zones to avoid; displaying the 3D model with the overlays to the user; updating, in real time, the displayed 3D model with the overlays as the surgical field of view changes from the received real-time video feed; extracting procedure data from the real-time video feed in real time; identifying, by the one or more processors in real time, a match between a predefined procedural landmark from the surgical procedure template and the extracted procedure data; and displaying visual confirmation on the display that user has matched the predefined procedural landmark.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows an example system for enabling surgical collaboration and providing surgical recommendations and templates.

FIG. 2 shows a schematic flowchart for operations that may be associated with a surgeon 201 performing operations associated with a poster.

FIG. 3 shows a schematic flowchart for operations that may be associated with a surgeon 301 performing operations associated with a reader.

FIG. 4 is a flowchart depicting an example method for performing surgical collaboration.

FIG. 5 is a flow diagram illustrating some steps or operations associated with the surgical collaboration method described in FIG. 4.

FIG. 6 is a flowchart depicting an example method for generating annotated surgical reports.

FIG. 7 is a flowchart depicting an example method for creating a repository of collaboration data.

FIG. 8 is a flowchart depicting an example method for responding to a request for feedback or guidance.

FIG. 9 is a flowchart depicting an example method for creating one or more surgical templates.

FIG. 10 shows a block diagram of a device that may be an example of any feasible device that may be configured to perform any operation described herein.

FIG. 11 is a flowchart of an example of training an image recognition algorithm.

FIG. 12 shows an example flowchart of the process of identifying a surgical procedure, as described herein.

FIG. 13 is a flowchart depicting operations for creating a surgical template.

FIG. 14 shows a schematic pipeline for using the surgical template of FIG. 13.

FIG. 15 shows a flowchart describing an algorithm to match the field of view from a video feed to a surgical template.

FIGS. 16 and 17 show views of example 3D navigational guidance that may be provided by execution of operations included in FIG. 14.

FIGS. 18 and 19 show an example view of positional guidance templates that may be provided using a surgical template.

### DETAILED DESCRIPTION

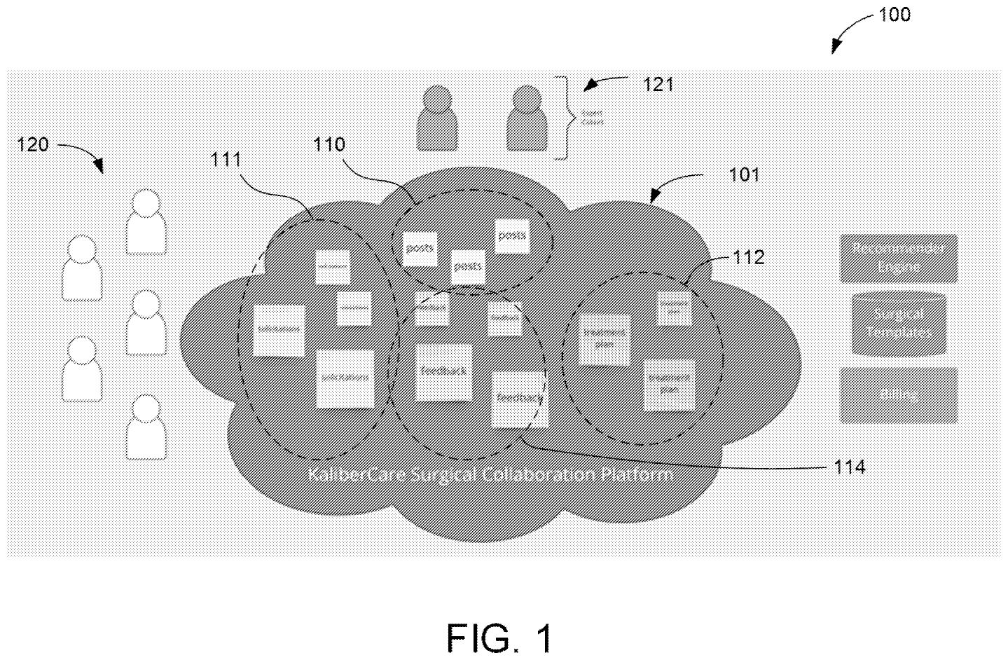

FIG. 1 shows an example system 100 for enabling surgical collaboration and providing surgical recommendations and templates. The system 100 may be implemented as a cloud-based collaboration platform. In some examples, a cloud 101 may include posts 110 from surgeons (or other medical professionals) 120. In some cases, the surgeons or other medical professionals may be members of an expert cohort 121 whose posts or feedback may provide more weight. The posts 110 may include a patient description, a solicitation for comments or guidance 111, and/or a treatment plan 112. The cloud 101 may also include feedback or advice 114 provided by surgeons 120 and/or the expert cohort 121. For example, a surgeon may ask for comments or guidance for an upcoming surgical procedure.

The treatment plans 112 may be provided by surgeons 120 and/or the expert cohort 121. In some cases, the treatment plans 112 may include treatment recommendations and surgical templates which may be determined from a repository.

The cloud 101 may include a surgeon skill repository. The surgeon skill repository may include surgical information determined from annotated surgery reports. In some examples, one or more artificial intelligence modules may be executed to process the annotated surgery reports to recognize anatomy, surgical actions, tools and implants, and the like. In some cases, the surgeon skill repository may be used to provide solicited feedback to the surgeon.

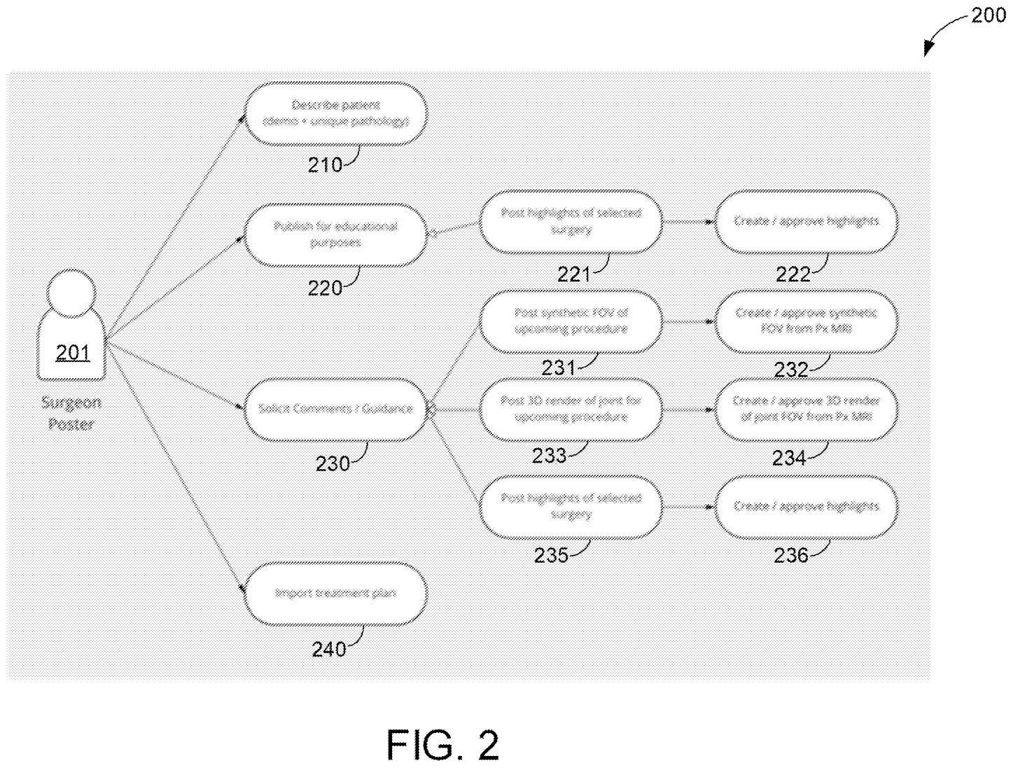

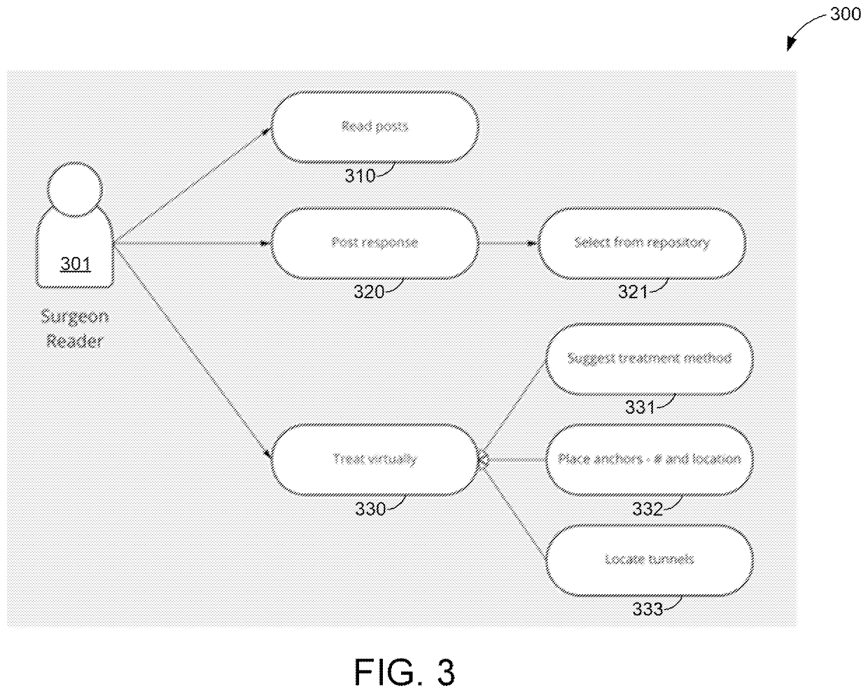

The system 100 can enable multiple surgeons to collaborate by enabling one or more surgeons to interact as a “poster” or a “reader.” In some cases, any surgeon may perform both poster and reader functions. A poster may be seeking feedback or advice regarding a surgical operation or procedure. A reader may be responding to one or more posts asking for feedback or advice. Although not shown, the system 100 may include one or more processors that may be configured to perform any operations described herein. Operations associated with the surgeon performing poster functions are described in more detail below in conjunction with FIG. 2. Operations associated with the surgeon performing reader functions are described in more detail below in conjunction with FIG. 3.

FIG. 2 shows a schematic flowchart 200 for operations that may be associated with a surgeon 201 performing operations associated with a poster. The schematic flowchart 200 includes four operations: 1) describing the patient 210, 2) publishing patient information 220, 3) soliciting comments and/or guidance 230, and 4) importing a treatment plan 240. Although only four operations are shown, in some embodiments, the schematic flowchart 200 may include any feasible number of operations. Operations associated with the schematic flowchart 200 may include uploading any feasible information associated with any steps.

Describing the patient 210 may include any feasible patient-specific information. In some variations, describing the patient 210 may include providing patient gender, weight or other patient demographic information. Describing the patient 210 may also include providing a description of any unique pathology for the patient. Pathologies may include a description of a tendon or ligament tear, a description of joint damage, a description of an orthopedic injury, a description of a damaged or injured vessel or lumen, or any other feasible pathology.

Publishing patient information 220 may include uploading patient information to a repository or data store. In some examples, the repository or data store may include virtual or cloud-based storage accessible through one or more network connections, including the Internet. Publishing patient information 220 may be for educational purposes. In some variations, the patient information may include any information associated with describing the patient 210 described herein. In some other variations, publishing patient information 220 may include posting highlights of a selected surgery 221. For example, the surgeon 201 may post or upload video associated with a patient considering or having undergone surgery. In some variations, the surgeon 201 may create and/or approve highlights 222 associated with the published patient information 220.

Soliciting comments and/or guidance 230 may include posting a synthetic (e.g., simulated) rendering of field of view (FOV) of an upcoming surgical procedure 231. Soliciting comments and/or guidance 230 may also include creating and/or approving highlights associated with the synthetic FOV 232.

In some examples, soliciting comments and/or guidance 230 may include posting a three-dimensional (3D) rendering of a joint associated with an upcoming surgical procedure 233. Soliciting comments and/or guidance 230 may also include creating and/or approving a 3D rendering of a joint FOV associated with the upcoming surgical procedure 234.

In some examples, soliciting comments and/or guidance 230 may include posting highlights of a selected surgery 235. Posting highlights of the selected surgery 235 may include creating and/or approving the highlights of the selected surgery 236.

Importing a treatment plan 240 may including uploading a proposed treatment plan, surgical plan, or operation for a patient.

FIG. 3 shows a schematic flowchart 300 for operations that may be associated with a surgeon 301 performing operations associated with a reader. The schematic flowchart 300 includes three operations: 1) reading the posts 310, 2) posting a response 320, and 3) treating virtually 330. Reading the posts 310 may include the surgeon 301 reading any information that may have been uploaded by the surgeon 201 of FIG. 2.

The surgeon 301 may post a response in response to reading a post in block 310. In some examples, posting a response 320 may include selecting information from a repository 321. The selected information may include information previously uploaded by the surgeon 301.

The surgeon 301 may treat a patient virtually 330. Treating a patient virtually 330 may include suggesting a treatment method 331. Treating a patient virtually 330 may include placing (or indicating where placement is to occur) anchors 332. The anchors may be associated with anchoring a ligament or other organ. Treating a patient virtually 330 may include locating tunnels 333. For example, the surgeon 301 may locate a tunnel on a patient's anatomy associated with an anterior cruciate ligament (ACL) reconstruction.

Additional steps, functions, and/or operations may be included with FIGS. 2 and 3. For example an authentication module may be included to ensure only qualified or permitted personnel can access the system 100. Billing and auditing modules may be included to enable participants to submit bills and allow a review (audit) of the overall system 100. In some variations, an application (“app”) may run on a tablet computing device, smart phone, or other computing device to enable a surgeon to interact as a poster (as described with respect to FIG. 2) or a reader (as described with respect to FIG. 3).

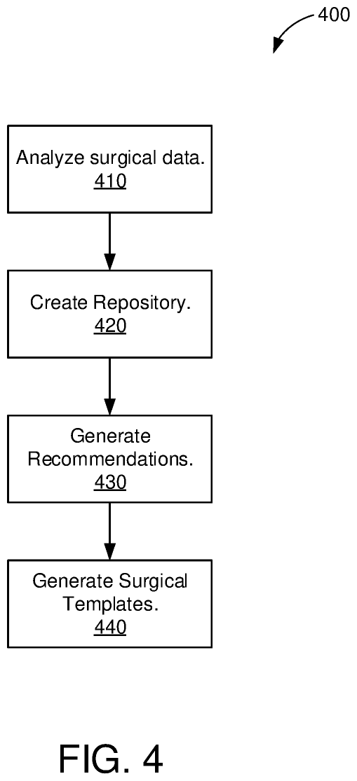

FIG. 4 is a flowchart depicting an example method 400 for performing surgical collaboration. Some examples may perform the operations described herein with additional operations, fewer operations, operations in a different order, operations in parallel, and some operations differently. The method 400 may enable a plurality of surgeons or other medical practitioners to collaborate, request and receive surgical advice or critique, and receive surgical guidance. The method 400 is described below with respect to system 100 of FIG. 1, however, the method 400 may be performed by any other suitable system or device.

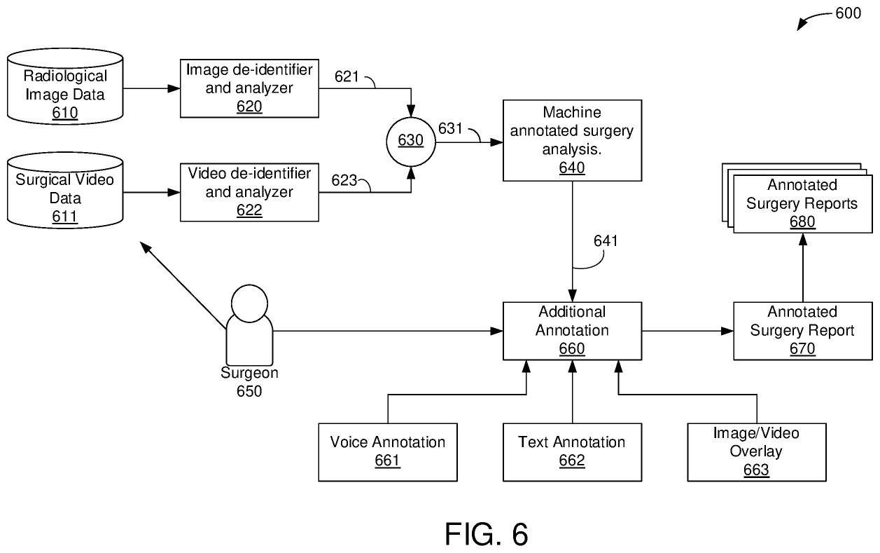

The method 400 begins in block 410 where surgical data is analyzed. For example, a surgeon can analyze and post (upload) surgical data associated with previous surgeries. The analyzed surgical data may include video data associated with a surgery and/or radiological procedures. In some examples, the surgical data may be annotated by the surgeon to highlight particular points of interest. Analyzed surgical data may be referred to surgical reports. Analysis of surgical data is described in more detail in conjunction with FIG. 6.

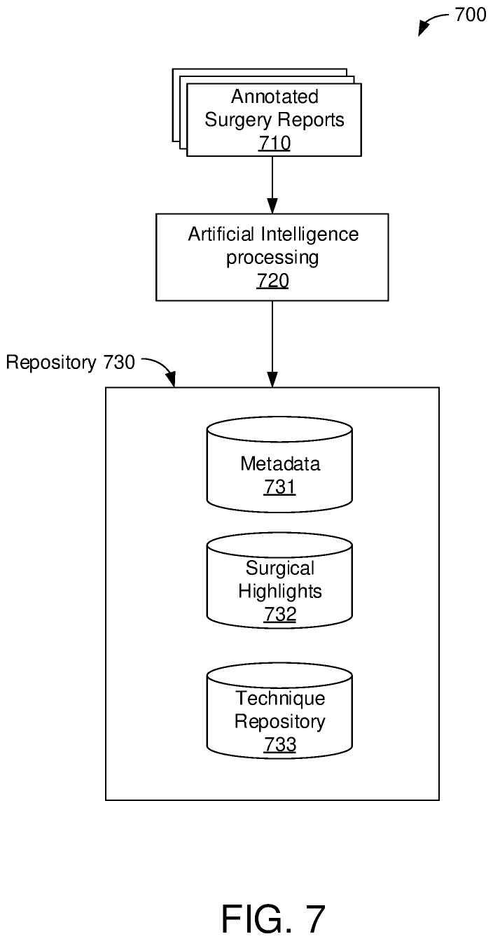

Next, in block 420 a repository is created. The repository may include the surgical reports (e.g., surgical data) that have been uploaded (posted) by a surgeon. In some variations, the uploaded surgical reports may be further processed and analyzed, in some cases by a processor executing a neural network to further annotate and analyze the surgical reports. Repository creation, including operations associated with neural networks, is described in more detail in conjunction with FIG. 7.

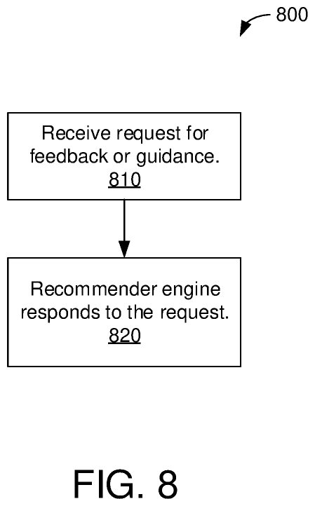

Next, in block 430 the system 100 generates recommendations. In some variations, the recommendations may be generated in response to a surgeon's request soliciting comments or guidance as described in FIG. 2. The generation of recommendations is described in more detail in conjunction with FIG. 8.

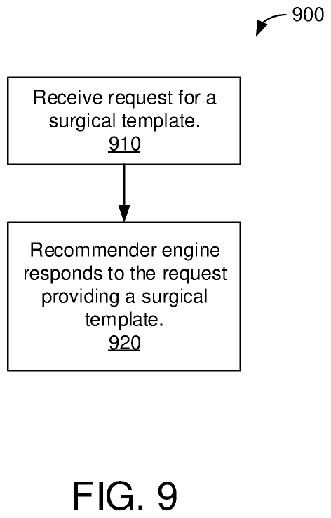

Next, in block 440 the system generates surgical templates. In some variations, the surgical templates may also be generated in response to a surgeon's request soliciting comments or guidance as described in FIG. 2. The generation of surgical templates is described in more detail in conjunction with FIG. 9.

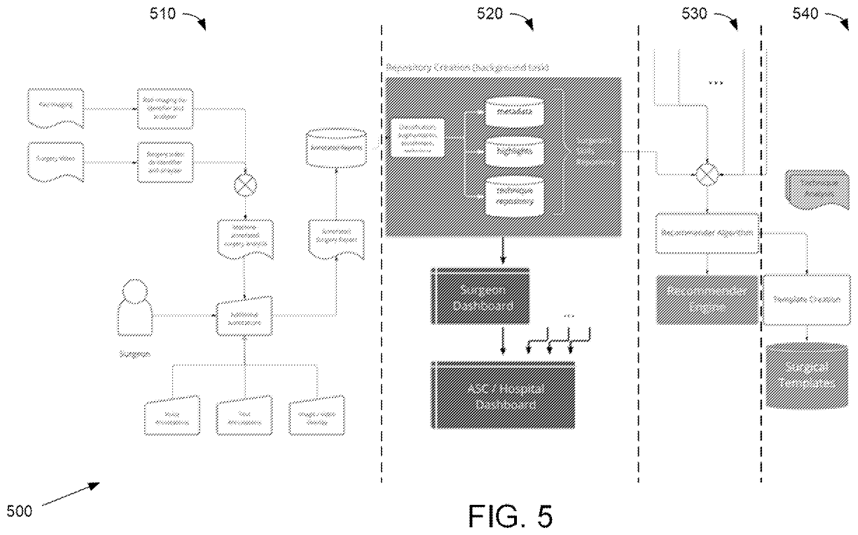

FIG. 5 is a flow diagram 500 illustrating some steps or operations associated with the surgical collaboration method described in FIG. 4. Steps or operations for analyzing surgical data (corresponding to block 410) are included within an analyzing surgical data section 510. Steps or operations for creating a repository (corresponding to block 420) are included within a creating a repository section 520. Steps or operations for generating recommendations (corresponding to block 430) are described within a generating recommendations section 530. Steps or operations for generating surgical templates (corresponding to block 440) are described within a generating surgical templates section 540.

FIG. 6 is a flowchart depicting an example method 600 for generating annotated surgical reports. The method 600 may generate one or more annotated surgical reports based on data made available by a surgeon or other clinician.

The method 600 may begin as a surgeon 650 uploads radiological image data 610 and/or surgical video data 611. In some examples, the surgeon 650 may upload the radiological image data 610 and/or surgical video data 611 to a network-based (cloud-based) storage device. The radiological image data 610 may include x-ray, ultrasound, or other non-visual image data.

Next, in block 620, the radiological image data is de-identified and analyzed. De-identification removes or redacts patient specific data or metadata that may identify or associate the radiological image data 610 with a specific patient. For example, information including patient name, patient number, medical number, or any other feasible information that may mark may identify or associate a particular patient with the radiological image data 610 may be removed or redacted. In this manner, a redacted radiological image data 621 may be shared without exposing or identifying a specific individual.

The surgical video data 611 may include actual video data from within a region that the surgeon is considering operating upon, for example, repairing or replacing a portion of the patient's anatomy. Next, in block 622 the surgical video data is de-identified and analyzed. De-identification removes or redacts patient specific data or metadata that may identify or associate the surgical video image data 611 with a specific patient. A redacted video image data 623 may be shared without exposing or identifying a specific individual.

In block 630, the redacted radiological image data 621 may be combined with redacted video image data 623 to generate combined image data 631. Next, in block 640, machine annotated surgery analysis is performed on the combined image data 631 to generate an initial annotated image data 641. Some examples of machine annotated surgery analysis may include rudimentary indication of video start and stop times.

Next, in block 660 the surgeon 650 may provide additional annotation to the initial annotated image data 641. For example, the surgeon 650 may add additional voice annotation 661 or text annotations 662 to any of the machine annotated surgery analysis 641. In some examples, the surgeon 650 may also add image or video overlay annotation 663.

After the surgeon 650 adds additional annotation, an annotated surgery report 670 is generated. The annotated surgery report 670 may include any or all of the data, information, and annotations mentioned herein. Multiple annotated surgery reports 670 may be collected to form the annotated surgery reports 680. In some variations, the annotated surgery reports 680 may be stored in a remote or network-accessible storage device. For example, the annotated storage reports 680 may be stored in a cloud-based storage device.

FIG. 7 is a flowchart depicting an example method 700 for creating a repository of collaboration data. The method 700 may create a repository of collaboration data from one or more annotated surgery reports. The method 700 is described below with respect to system 100 of FIG. 1, however, the method 700 may be performed by any other suitable system or device.

The method begins in block 710 as the system 100 collects, obtains, or otherwise accesses annotated surgery reports 710. The annotated surgery reports 710 may be another example of the annotated surgery reports 680 of FIG. 6.

Next, in block 720, the system 100 performs artificial intelligence processing 720 on the annotated surgery reports 710 to create a repository 730. Performing artificial intelligence processing may include executing any number of trained neural networks using the annotated surgery reports 710 as input. The artificial intelligence processing 720 may generate metadata 731, surgical highlights 732, and a technique repository 733. Although only three items are described, in other embodiments, the artificial intelligence processing 720 may generate any feasible number of items in the repository 730.

The metadata 731 may include any number of medical terms, descriptions, findings or the like that may enable a clinician to search for and/or identify an annotated report. Examples of metadata 731 may include patient information (e.g., demographic information such as age, gender, weight and the like), radiological findings, clinical notes, patient diagnosis, clinical notes, or any other feasible identifiers or descriptors. The metadata 731 may not be directly visible to a clinician. The surgical highlights 732 may include highlights of surgical videos that are included within any of the annotated surgery reports 710. For example, a surgical highlight of an ACL operation may include portions of the video where a replacement ligament is anchored to a bone. The technique repository 733 may include portions of the annotated surgery reports 710 that have been determined to demonstrate and/or describe surgical techniques that address any number of surgical cases.

The artificial intelligence processing 720 may include any number of feasible neural networks that may generate items for any portion of the repository 730. For example, the system 100 (or one or more processors within the system 100) may execute a neural network trained to understand and/or recognize an anatomical area or surgical area from within any of the annotated surgery reports 710. In some variations, execution of a neural network may process de-identified radiological image data and/or de-identified video image data to recognize an anatomical region and determine a basic context for a surgical procedure. In addition, the system 100 may annotate and associate the de-identified radiological image data and/or the de-identified video image data with information indicating a particular anatomical area.

In another example, the system 100 (or one or more processors within the system 100) may execute a neural network trained to recognize different surgery stages. In some variations, execution of a neural network may process de-identified radiological image data and/or de-identified video image data to recognize an anatomical region and determine whether surgery has begun or is proceeding within a surgical area. In some cases, the neural network may be trained to detect and/or identify progress associated with different surgical procedures. In some variations, a neural network may be trained to determine whether or not a surgery stages has or has not advanced.

In another example, the system 100 (or one or more processors within the system 100) may execute a neural network trained to recognize surgical tools and/or surgical implants. In some variations, execution of a neural network may process de-identified radiological image data and/or de-identified video image data to recognize when and where tools and/or implants are used. In some variations, execution of the neural network may also recognize sutures and/or anchors within a surgical area.

In some examples, a higher level analysis may be performed on the de-identified radiological image data and/or the de-identified video image data. For example, the system 100 (or one or more processors within the system 100) execute a neural network trained to detect when tools and/or implants are placed or used within the context of one or more surgical actions (procedures). In some variations, execution of the neural network may also match particular portions of de-identified radiological image data and/or de-identified video image data with a surgical action involving a particular surgical tool.

In another example, the system 100 (or one or more processors within the system 100) may execute a neural network trained to recognize scene changes within any de-identified radiological image data and/or de-identified video image data. In some variations, execution of the neural network can identify and highlight significant surgical actions and/or procedures within any identified scene.

Notably some of all of the above-noted actions and procedures may run autonomously with respect to any surgeon, clinician, or user. For example, the system 100 may perform any feasible action or execute any neural network without any involvement or participation from any personnel. In some variations, the autonomous programs may be said to be “running in the background.” In this manner, the artificial intelligence processing 720 may cause execution of one or more neural networks to generate metadata 731, surgical highlights 732, and/or technique repository 733 information from the annotated surgery reports 710. The information in the repository 730 may be used to generate expert recommendations and create surgical templates. These actions are described in more detail below with respect to FIGS. 8 and 9.

FIG. 8 is a flowchart depicting an example method 800 for responding to a request for feedback or guidance. In some cases, a response to the request may be in the form of a recommendation. In some examples, the method 800 may respond by providing one or more annotated surgical reports. The method 800 is described below with respect to system 100 of FIG. 1, however, the method 800 may be performed by any other suitable system or device.

The method 800 begins in block 810 where the system 100 receives a request for feedback or guidance. For example, referring to FIG. 2, the system 100 may receive solicitation for comments and/or guidance from the surgeon 201 performing operations associated with a poster. For instance, the surgeon 201 may be preparing for an atypical (with respect to the surgeon 201) operation and may therefore be seeking an expert opinion regarding surgical procedures.

After receiving the request, the method 800 proceeds to block 820 where a recommender engine (in some examples using one or more recommender algorithms) may respond to the request. For example, the system 100 (or one or more processors within the system 100) may execute a neural network trained to provide information from the repository 730 of FIG. 7 associated with the request to the surgeon 201. In some examples, the neural network may be trained to suggest an item of data within the repository 730 most appropriate for a given situation. For example, the neural network may be trained to match one or more terms within the request with one or more metadata items that may be associated with a surgery report. In some examples, the metadata items may be determined as described herein with respect to FIG. 7. In some variations, the system 100 may consult a historical database and determine which annotated surgical report is most relevant to the request based on historical interactions or requests. Training of the neural network may be based, at least in part, on observing interactions between any number of surgeons regarding similar surgical subject matter and requests.

In another example, the recommender engine may suggest surgical highlights from a peer surgeons'repository (which may be stored within the repository 730). The responder engine may determine metadata that is associated with the request (received in block 810). The metadata may include patient information, radiological findings, clinical notes, and the like. The responder engine may use metadata associated with the request to find corresponding metadata 731 within the repository 730. In this manner, the responder engine may find and/or suggest surgical highlights (e.g., a highlight video including a surgical procedure) from annotated surgical reports that may be relevant for the surgeon 201.

FIG. 9 is a flowchart depicting an example method 900 for creating one or more surgical templates. In some examples, the method 900 may respond with one or more surgical templates to guide surgeon in an interoperative setting. The method 900 is described below with respect to system 100 of FIG. 1, however, the method 900 may be performed by any other suitable system or device.

The method 900 begins in block 910 where the system 100 receives a request for a surgical template. For example, referring to FIG. 2, the system 100 may receive solicitation for comments and/or guidance from the surgeon 201 performing operations associated with a poster. In some variations, the system 100 may receive a request for a surgical template that may be used in an interoperative setting.

After receiving the request, the method 900 proceeds to block 920 where the recommender engine may respond to the request for a surgical template. For example, the system 100 (or one or more processors within the system 100) may execute a neural network trained to provide video highlights of an operation corresponding the request received in block 910. In some instances, the highlight video may include expert-panel recommended techniques for use with a specific patient. Training for the neural network (such as described in U.S. patent application Ser. No. 17/918,873, filed Oct. 13, 2022 and PCT application US2021/027109 filed Apr. 13, 2021 all commonly assigned, the disclosures of which are incorporated by reference herein in their entireties) may be based on interaction between other surgeons. In some examples, interactions between and/or from the expert cohort 121 of FIG. 1 may be provided more “weight” by the recommender engine in determining a surgical template to provide in response to the request. That is, the recommender engine may generate a surgical template based on weighted recommendations from surgical peers (e.g., the surgeons 120) and experts (the expert cohort 121). In some variations, since the neural network is trained by expert surgeons from the expert cohort 121, then the surgical template determined by the system may be considered as expert panel recommendations. Execution of any of the neural networks described herein may include matching metadata terms included within the request received in block 910 with metadata terms included within the metadata 731 of the repository 730 of FIG. 7.

In some examples, the surgical template may include treatment details including, but not limited to, anchors for rotator cuff repair for a given level of bone loss, or any other surgical repair. In some other examples, the surgical template may include indicating a location of a tunnel placement associated with ACL reconstruction surgery.

In another example, the surgical template may be decomposed into a number of specific surgical actions. For example, as a surgeon performs a surgery, the surgeon can retrieve a surgical template and that may include the overlays from the recommendations. The overlays may be displayed onto or over a surgical field of view. The neural network may be trained to recognize anatomy and may run on the real-time surgery video feed and match surgical context from the surgical template and project a scaled overlay onto the field of the surgeon's view in the form of a colored mask. The surgeon may consult the recommendation and can deactivate the overlay once the surgeon has determined an appropriate way to treat the patient.

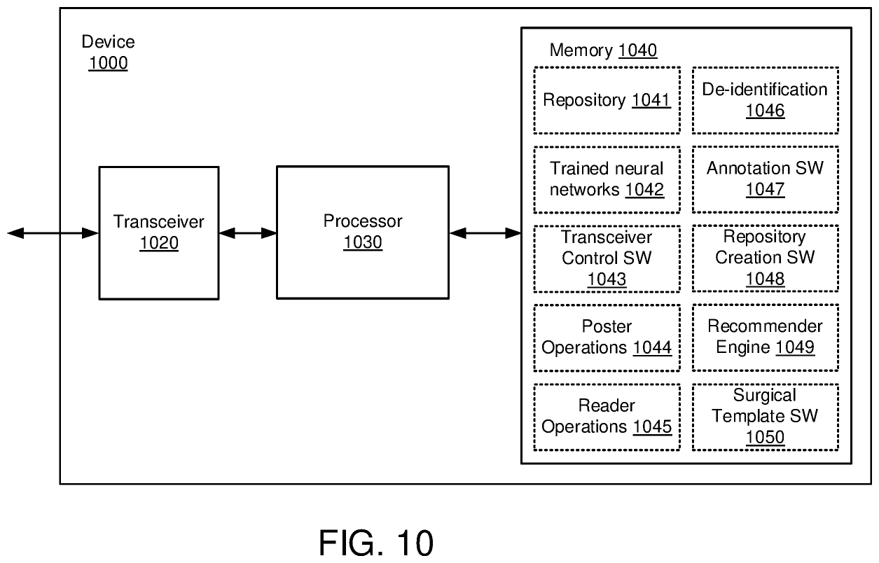

FIG. 10 shows a block diagram of a device 1000 that may be an example of any feasible device that may be configured to perform any operation described herein. The device 1000 may include a transceiver 1020, a processor 1030, and a memory 1040.

The transceiver 1020, which is coupled to the processor 1030, may be used to interface with any other device. For example, the transceiver 1020 may include a wireless and/or a wired transceiver configured to transmit and/or receive data according to any technically feasible protocol. In some variations, the transceiver 1020 may include a wired ethernet interface. In some other variations, the transceiver 1020 may include a wireless interface that may communicate via Bluetooth, Wi-Fi (e.g., any IEEE 802.11 compliant implementation), Long Term Evolution (LTE) standard, or the like. In some embodiments, the transceiver 1020 may be coupled to a network, such as the Internet, thereby coupling the device 1000 to any other device or service through the network.

The processor 1030, which is also coupled to the memory 1040, may be any one or more suitable processors capable of executing scripts or instructions of one or more software programs stored in the device 1000 (such as within memory 1040).

The memory 1040 may include a repository 1041 that may be used to locally store surgical video data, radiological image data, metadata, surgical highlights, technique repository, patient information, patient diagnosis, or the like. The repository 1041 may be an example implementation of the repository 730 of FIG. 7.

The memory 1040 may also include one or more trained neural networks 1042. The trained neural networks 1042 may be executed by the processor 1030 to perform any feasible, artificial intelligence-related function. Operations of various trained neural networks have been described herein. Thus, the various trained neural networks may be stored as the trained neural networks 1042.

The memory 1040 may also include a non-transitory computer-readable storage medium (e.g., one or more nonvolatile memory elements, such as EPROM, EEPROM, Flash memory, a hard drive, etc.) that may store the following software modules:
a transceiver control software (SW) module 1043 to control the transceiver 1020;
a poster operations module 1044 to control and enable posting operations;
a reader operations module 1045 to control and enable reader operations;
a de-identification module 1046 to redact confidential patient information;
an annotation SW module 1047 to annotate video or image files;
a repository creation SW module 1048 to create and store repository data;
a recommender engine 1049; and
a surgical template SW module 1050.

Each software module includes program instructions that, when executed by the processor 1030, may cause the device 1000 to perform the corresponding function(s). Thus, the non-transitory computer-readable storage medium of memory 1040 may include instructions for performing all or a portion of the operations described herein.

The processor 1030 may execute the transceiver control SW module 1043 to transmit and/or receive data through the transceiver 1020. In some examples, the transceiver control SW module 1043 may include software to control wireless data transceivers that may be configured to transmit and/or receive wireless data. In some cases, the wireless data may include Bluetooth, Wi-Fi, LTE, or any other feasible wireless data. In some other examples, the transceiver control SW module 1043 may include software to control wired data transceivers. For example, execution of the transceiver control SW module 1043 transmit and/or receive data through a wired interface such as, but not limited to, a wired Ethernet interface.

The processor 1030 may execute the poster operations module 1044 to enable a clinician (e.g., a surgeon or other practitioner) to “post” questions, comments, image data and the like to a system, such as the system 100 of FIG. 1. In some examples, execution of the poster operations module 1044 may enable or perform one or more tasks as described in FIG. 2.

The processor 1030 may execute the reader operations module 1045 to enable a clinician to “read” postings, comments and the like with respect to the system 100 of FIG. 1. In some examples, execution of the reader operations module 1045 may enable or perform one or more tasks as described in FIG. 3.

The processor 1030 may execute the de-identification module 1046 to remove patient identifying information from uploaded video data and/or radiological information data. In some examples, execution of the de-identification module 1046 to redact any sensitive patient information from any feasible file or document.

The processor 1030 may execute the annotation SW module 1047 to annotate any feasible surgical report. For example, execution of the annotation SW module 1047 may enable a surgeon to add voice, text, or image notations to video or radiological image data. In some other examples, execution of the annotation SW module 1047 may perform some or all of the annotation operations described herein, such as those described with respect to FIG. 6.

The processor 1030 may execute the repository creation SW module 1048 to add any feasible surgical data to a repository, such as the repository 730 of FIG. 7 or the repository 1041. In some examples, execution of the repository creation SW module 1048 may cause the processor to further execute one or more trained neural networks (within the trained neural networks 1042) to add one or more items to the repository 730 and/or the repository 1041. Some example neural networks are described within, but not limited to, FIG. 7.

The processor 1030 may execute the recommender engine 1049 to respond to one or more requests for feedback and/or guidance. For example, execution of the recommender engine 1049 may further execute one or more trained neural networks (within the trained neural networks 1042) to suggest an item with a repository in response to a feedback or guidance request. In some examples, execution of the recommender engine 1049 may suggest one or more surgical highlights to provide in response to a request. In another example, execution of the recommender engine may determine metadata that is associated with the request. The responder engine may use metadata associated with the request to find corresponding metadata within the repository 730. In some examples, the recommender engine 1049 may perform any operations described in conjunction with, but not limited to, FIGS. 8 and 9.

The processor 1030 may execute the surgical template SW module 1050 to generate one or more surgical templates in response to a request. For example, execution of the surgical template SW module 1050 may further execute one or more trained neural networks to provide a highlight video. The highlight video may include expert-panel recommended techniques for use with a specific patient. In some examples, the surgical template SW module 1050 may perform any operation described in conjunction with, but not limited to, FIG. 9.

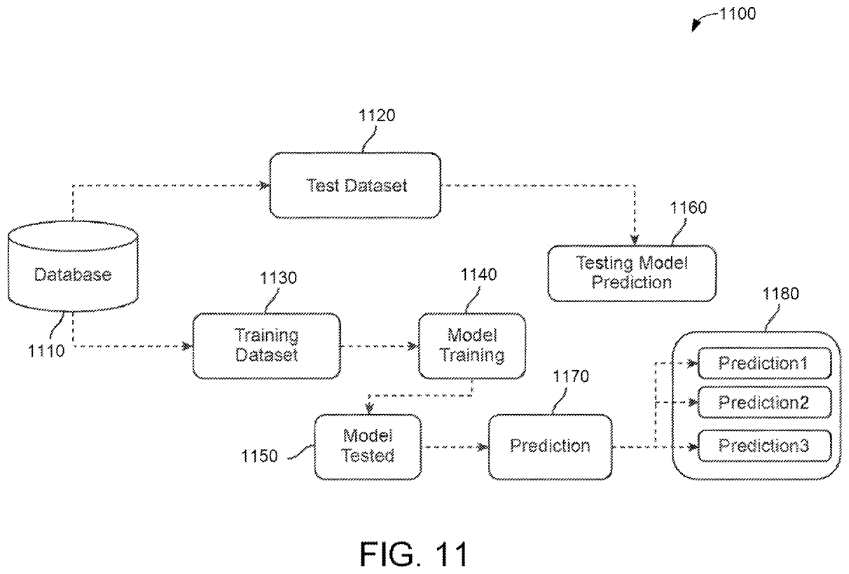

FIG. 11 is a flowchart of an example of training an image recognition algorithm. An AI training method 1100 may comprise a dataset 1110. The dataset 1110 may comprise images of a surgical tool, an anatomical structure, an anatomical feature, a surgical tool element, an image acquired from a video feed of an arthroscope, a portal of a surgery, a region of a surgery, etc. The dataset may further comprise an imaged that has been edited or augmented using the methods described hereinbefore. The images in the dataset 1110 may be separated into at least a test dataset 1120 and a training dataset 1130. The dataset 1110 may be divided into a plurality of test datasets and/or a plurality of training datasets. At a model training step 1140, a training dataset may be used to train an image recognition algorithm. For example, a plurality of labeled images may be provided to the image recognition algorithm to train an image recognition algorithm comprising a supervised learning algorithm (e.g., a supervised machine learning algorithm, or a supervised deep learning algorithm). Unlabeled images may be used to build and train an image recognition algorithm comprising an unsupervised learning algorithm (e.g., an unsupervised machine learning algorithm, or an unsupervised deep learning algorithm). A trained model may be tested using a test dataset (or a validation dataset). A test dataset may comprise unlabeled images (e.g., labeled images where a label is removed for testing a trained model). The trained image recognition algorithm may be applied to the test dataset and the predictions may be compared with actual labels associated with the data (e.g., images) that were removed to generate the test dataset in a testing model predictions step 1160. A model training step 1140 and a testing model predictions step 1160 may be repeated with different training datasets and/or test datasets until a predefined outcome is met. The predefined outcome may be an error rate. The error rate may be defined as one more of an accuracy, a specificity, or a sensitivity or a combination thereof. The tested model 1150 may then be used to make a prediction 1170 for labeling features in an image from an imaging device (e.g., an arthroscope) being used in the course of a medical procedure (e.g., arthroscopy). The prediction may comprise a plurality of predictions 1180 comprising a region of a surgery, a portal of the surgery, an anatomy, a pathology, a tool, an action being performed, a procedure being performed, etc.

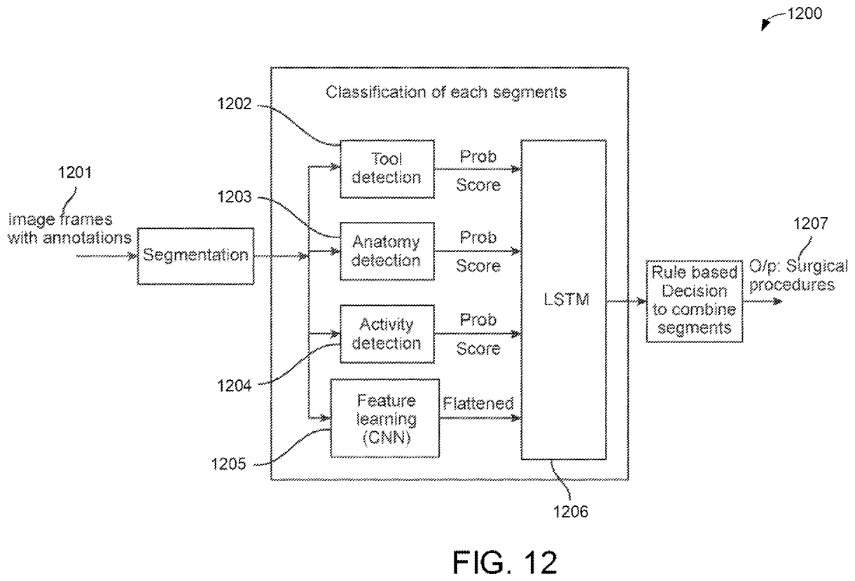

FIG. 12 shows an example flowchart of the process of identifying a surgical procedure 1200, as described herein. Image frames with annotations 1201 may be received and segmented into one or more segments using one or more classifier models. The classifier models may comprise a tool recognition model 1202, an anatomy detection model 1203, an activity detection model 1204, or a feature learning model 1205. The outputs from the one or more classifiers may be combined using a long short term memory (LSTM) 1206. LSTM is an artificial recurrent neural network (RNN) classifier that may be used to predict based on image recognition at one moment compared to what has been recognized prior. In other words, LSTM may be used to generate a memory of a context of the images being processed, as described herein. The context of the images is then used to predict a stage of the surgery comprising a surgical procedure. A rule-based decision to combine the classified segments into one image may then be processed to identify/predict a surgical procedure 1207 being performed.

Another aspect of the invention provides a system for implementing a hierarchical pipeline for guiding an arthroscopic surgery. The system may comprise one or more computer processors and one or more non-transitory computer-readable storage media storing instructions that are operable, when executed by the one or more computer processors, to cause the one or more computer processors to perform operations. The operations may comprise (a) receiving at least one image captured by an interventional imaging device; (b) identify one or more image features of a region of treatment or a portal of entry in the region based on at least one upstream module; (c)) activating a first downstream module to identify one or more image features of an anatomical structure, or a pathology based at least partially on the identified one or more image features in step (b); (d) activating a second downstream module to identify one or more image features of a surgical tool, a surgical tool element, an operational procedure or action relating to the arthroscopic surgery based at least partially on the identified one or more image features in step (b); (e) labeling the identified one or more image features; and displaying the labeled one or more image features in the at least one image continuously to an operator in the course of the arthroscopic surgery. The at least one upstream module may comprise a first trained image processing algorithm. The downstream module may comprise a second trained image processing algorithm. The second downstream module may comprise a third trained image processing algorithm.

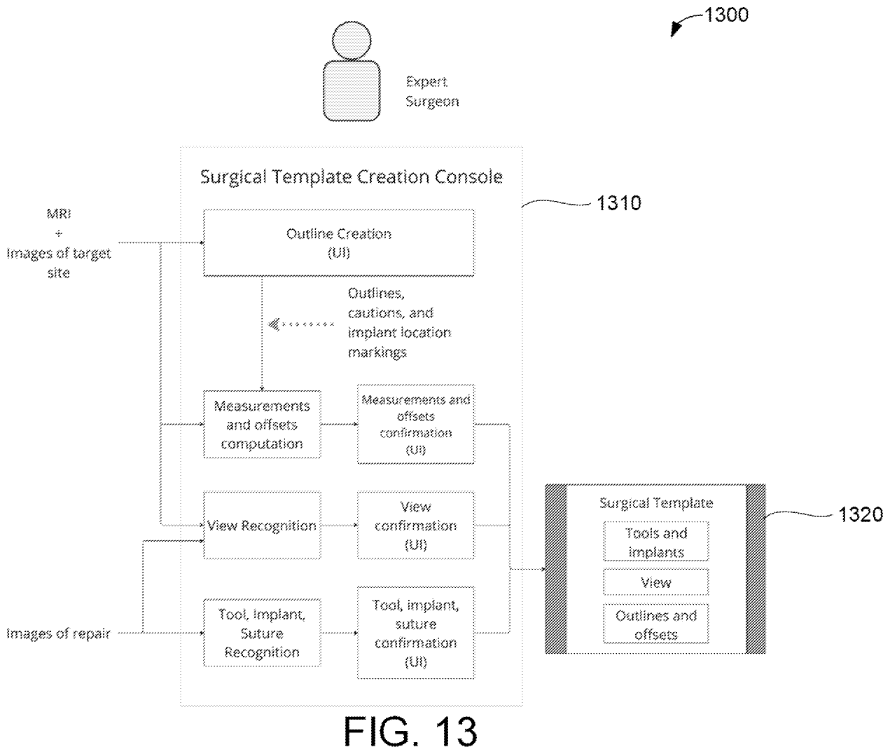

FIG. 13 is a flowchart depicting operations for creating a surgical template 1300. In general, creating a surgical template may correspond to actions described herein associated with FIG. 9. In other words, creating the surgical template may correspond to a surgeon acting as a poster seeking feedback or advice for a surgical operation or procedure. In some examples, radiological and/or surgical images from actual procedures may form at least part of the input into the creating the surgical template. The process may be divided into the following tasks that may be performed by a surgical template creation console 1310.

### Target Site Fixation.

The requesting surgeon may select representative images showing the target sites for the procedure. For example, for an ACL reconstruction, a surgeon may select a femoral condyle and the tibial plateau. The surgical template creation console 1310 applies anatomy and view recognition algorithms on the images of the selected images of the target sites. The surgeon may then be prompted to validate the recognized view. Once confirmed, information about the view is added to the template.

### Procedure Planning

In some examples, the surgeon can use a combination of still images from the procedure and the patient's preoperative MRI to mark offsets, recommended implant sites, or the like. The surgical template creation console 1310 matches the radiological (x-ray, MRI, or the like) images to the still images from the surgical procedure.

Radiological images establish the ground truth for the physical dimensions of the structures seen in the image. When the anatomical structures are matched from the radiological and the imaging modalities, the physical dimensions are translated to the dimensions of the corresponding structures, full or partial, seen in the surgery images. The offsets and the dimensions, shown in relation to the dimensions of well-recognized structures, ex. Humeral head in the shoulder, the femoral condyle in the knee, etc. Once the surgeon confirms the offsets (offsets of locations of implants and/or anchors) and the measurements, this information is also loaded into the surgical template 1320.

### Tool and Implant Fixation

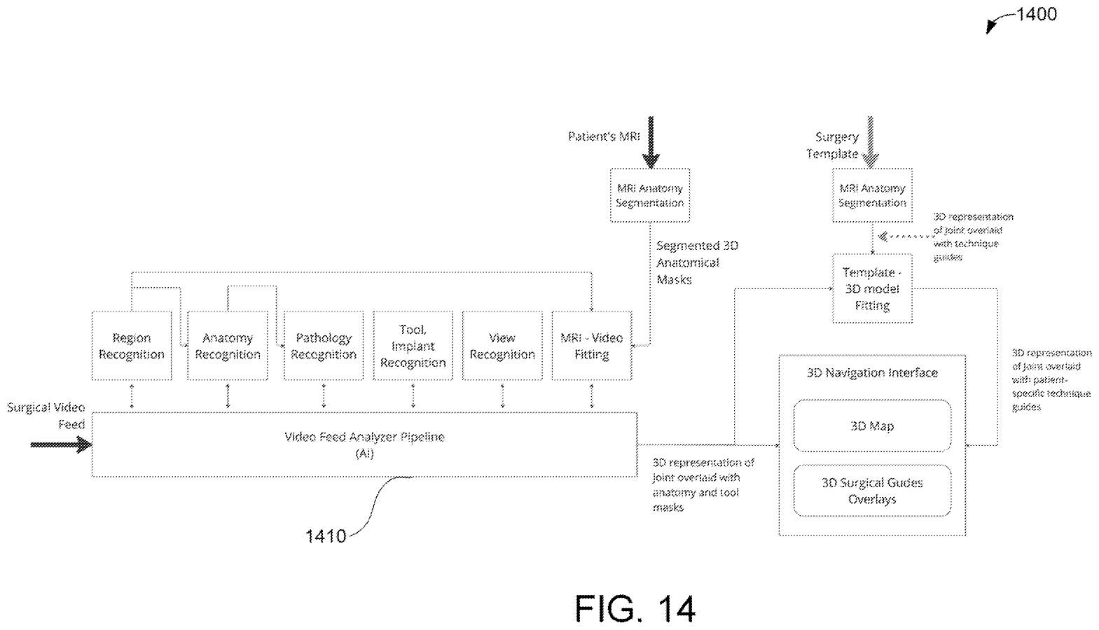

The surgeon uses a combination of still images from the procedure showing various salient points during the repair process. The images of the repair are analyzed by an artificial intelligence (AI) pipeline (as described below in FIG. 14). Once the surgeon validates the set of recognized tools, implants, and the views provided by the AI pipeline, this information is added to the surgical template 1320.

The AI pipeline (in some examples operations any AI pipeline described herein may be provided by a processor executing a trained neural network) may analyze various aspects of the repair. These attributes, include but are not limited to: determine a relative position of the tools and implants with respect to known anatomical structures, determine angles, such as approach angles of drills which deliver implants to bony structures, and determine a presence of pathology and other anatomical structure at the target site. All of these determined attributes may be saved to the surgical template 1320.

### Use of Surgical Templates During Surgery

FIG. 14 shows a schematic pipeline 1400 for using the surgical template 1320 developed in FIG. 13. During the surgery, the surgical template 1320 is imported and used to provide guidance to the surgeon. In general, an AI pipeline can analyze a video feed in real time, and match the images in the field of view to segmented images from the patient's MRI. These matched images are then used to determine and assign physical dimensions to the images in the field of view.

The surgical template 1320 is imported. Through a video feed analyzer pipeline 1410, the surgeon is alerted when the field of view matches the view specified in the surgical template 1320. Once the surgeon confirms that the view has been realized (matches), the surgical template 1320 can be activated. At this point, a 3D model that matches the field of view, the patient's MRI, and/or other corresponding images from the template is determined. This algorithm is described in detail in the following section.

The matching described herein provides a mapping between the recommendations in the surgical template 1320 and field of view. Various instructions, such as offsets, measurements, zones to avoid, etc., are mapped from the surgical template 1320 to the field of view along with corresponding anatomical structures. One or more overlays are now visualized (displayed) in the field of view along with the real time video feed. The overlays and video feed are used by the surgeon to perform the procedure. The surgeon can choose to follow or ignore the recommendations provided by the surgical template 1320 through the one or more overlays.

A real-time processing engine executing the pipeline 1400 can also matches the views stipulated in the surgical template 1320 to the views achieved in the field of view of the surgical video feed and provide visual confirmation when the surgeon achieves the intermediate views at critical stages in the surgery.

In some examples, the view recognition engine can alert the surgeon that he/she is not properly positioned to deliver a given implant. Improper positioning could result in the delivery of anchors at incorrect angles. In other cases, a failure for the surgeon to achieve a proper view could result in failures to achieve proper implant positioning.

Other cues could also be provided to aid the surgeon. For example, a view classification can analyze a pathology and anatomical structures in the field of view and can provide a different kind of alert indicating that the target site might not have been prepared to the specifications in the surgical template 1320. The surgeon could again choose to ignore the alert after determining that this site is appropriate for the patient, overriding the guidance from the surgical template 1320.

### Field of View Template Matching Algorithm

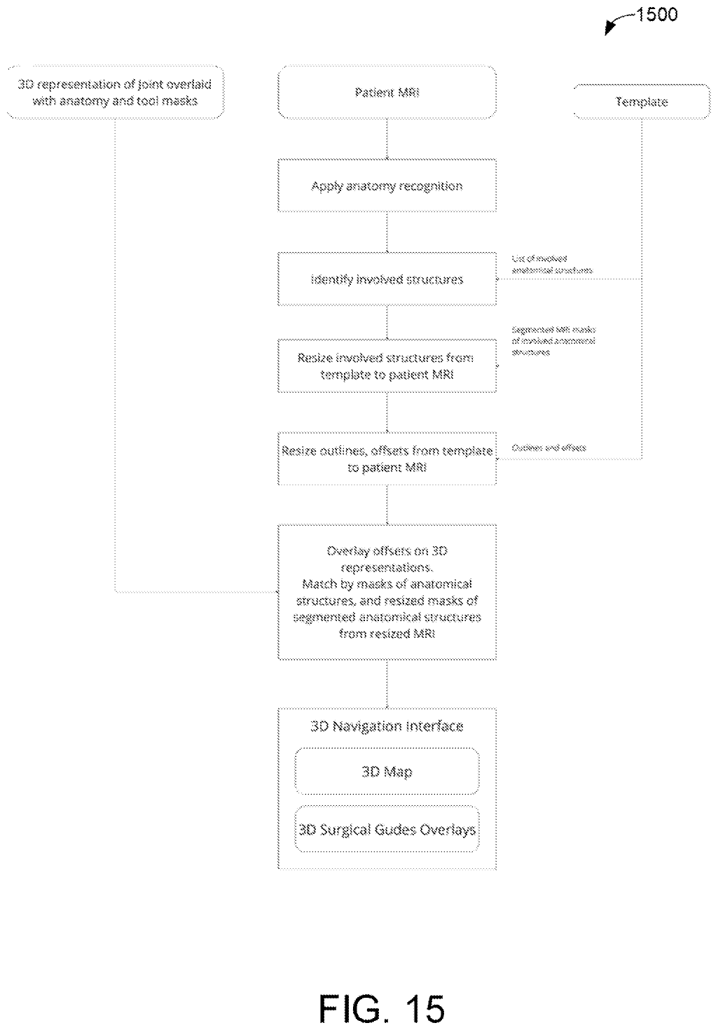

FIG. 15 shows a flowchart 1500 describing an algorithm to match the field of view from a video feed to a surgical template (such as the surgical template 1320 of FIG. 13). It should be noted that the matching occurs in the realm of radiological (x-ray, MRI and the like) images. Physical dimensions of anatomical structures seen in the field of view are scaled to the patient's MRI. The offsets and layout in the surgical template 1320 are specified in relation to major, procedure specific, anatomical structures seen in the MRI. The relative offsets are mapped to physical dimensions by matching the corresponding reference anatomical structure in the patient's MRI and obtaining its dimension.

Once the physical dimensions are determined, the MRI-field of view matching algorithms denoted in FIG. 15 estimates the offsets and implants positions by matching the AI-generated, segmented, masks of corresponding anatomical structures.

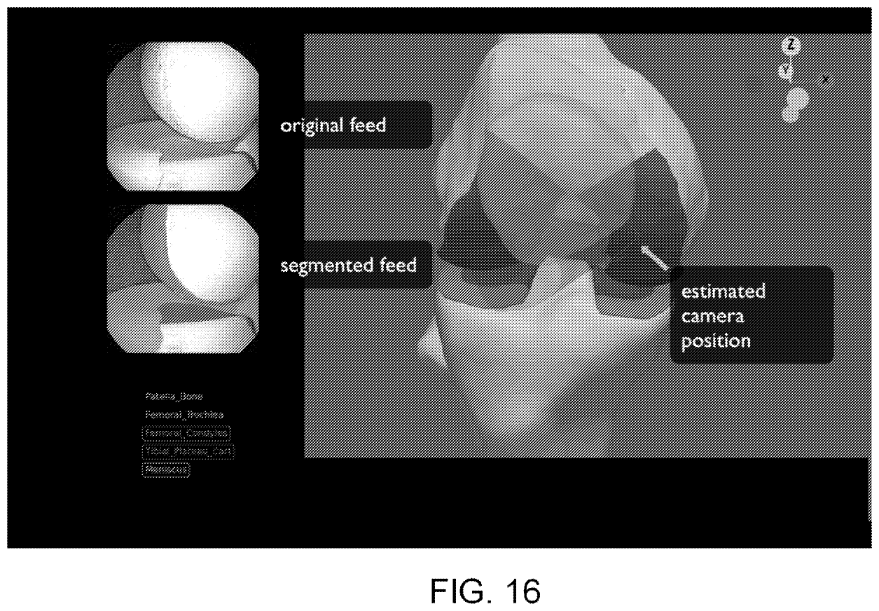

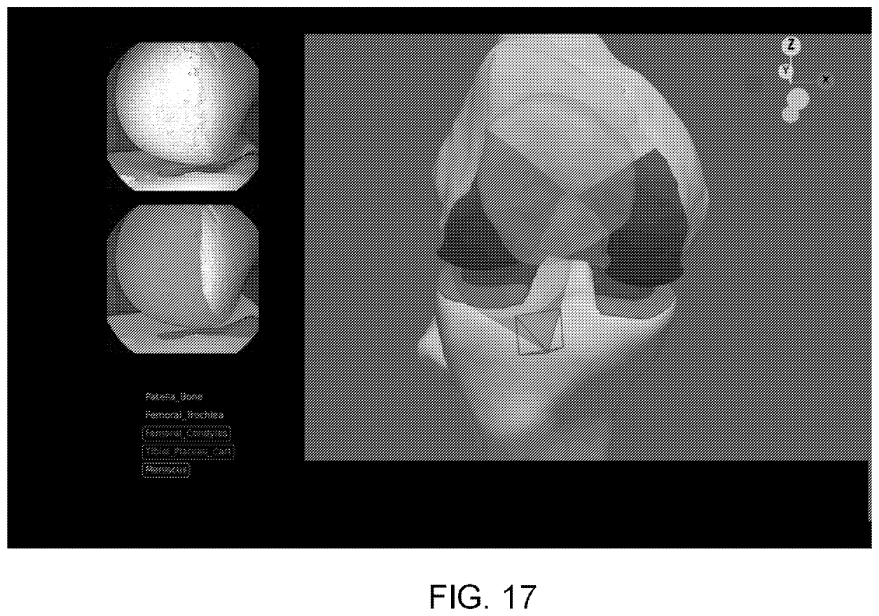

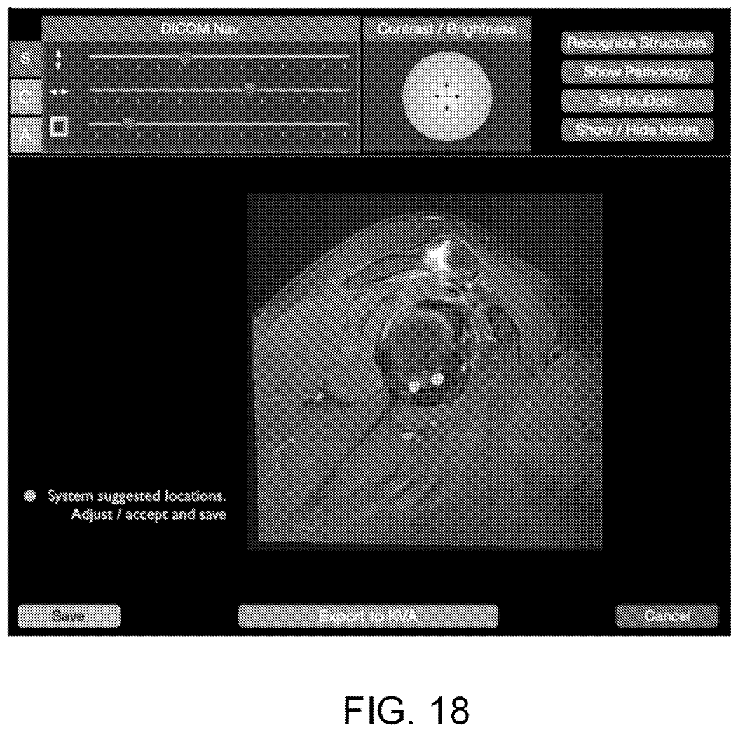

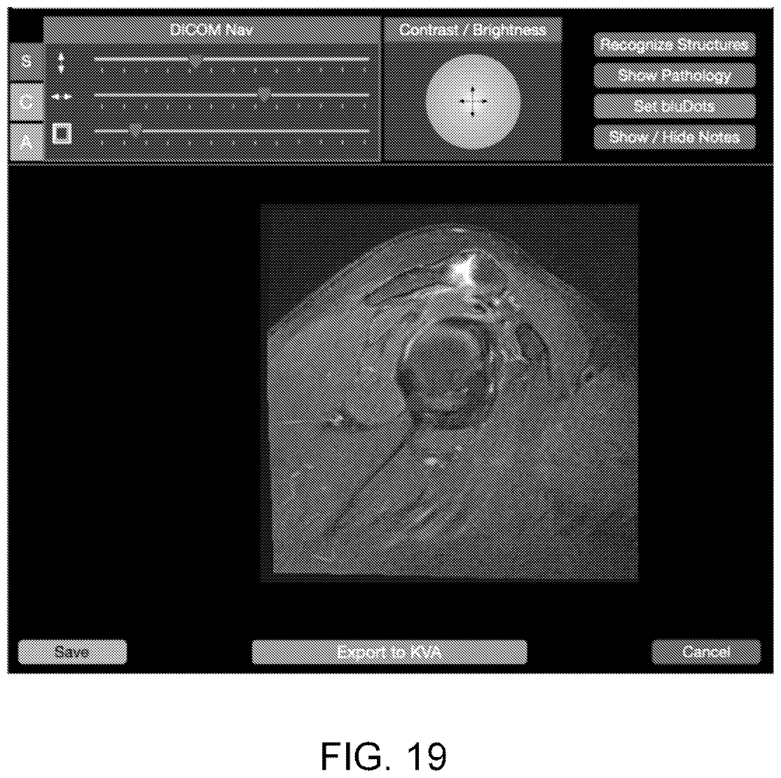

FIGS. 16 and 17 show views of example 3D navigational guidance that may be provided by execution of operations included in FIG. 14. FIGS. 18 and 19 show an example view of positional guidance templates that may be provided using the surgical template 1320.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control or perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A system for assisting in a surgical procedure, the system comprising:
one or more processors;
a memory coupled to the one or more processors, the memory storing computer-program instructions, that, when executed by the one or more processors, perform a computer-implemented method comprising:
receiving a real-time video feed of a surgical field of view;
matching a field of view specified in a surgical procedure template to the surgical field of view to generate a 3D model of the surgical field of view;
activating the surgical procedure template after a user confirms that the field of view of the surgical procedure template matches the surgical field of view;
transferring overlays from the activated template to the 3D model, wherein the overlays comprise one or more of: offsets, measurements, or zones to avoid;
displaying the 3D model with the overlays to the user;
updating, in real time, the displayed 3D model with the overlays as the surgical field of view changes from the received real-time video feed;
extracting procedure data from the real-time video feed in real time;
identifying, by the one or more processors in real time, a match between a predefined procedural landmark from the surgical procedure template and the extracted procedure data; and
displaying visual confirmation on the display that user has matched the predefined procedural landmark.

2. The system of claim 1, wherein the computer-implemented method further comprises: matching one or more pre-surgical patient scans to the 3D model.

3. The system of claim 2, wherein the one or more pre-surgical patient scan comprises MRI scans.

4. The system of claim 1, wherein updating the displayed 3D model with the overlays as the surgical field of view changes from the received real-time video feed further comprises updating the overlays.

5. The system of claim 1, wherein the procedure data comprises the surgical field of view or a modified version of the field of view.

6. The system of claim 1, wherein the procedure data comprises one or more of: visual data of implant position, surgical tool position, or anatomy orientation.

7. The system of claim 1, wherein the computer-implemented method further comprises: identifying, by the one or more processors in real time, a mismatch between the predefined procedural landmark from the surgical procedure template and the extracted procedure data.

8. The system of claim 7, wherein the computer-implemented method further comprises: displaying visual confirmation on the display that user has not matched the predefined procedural landmark.

9. The system of claim 1, wherein the computer-implemented method further comprises: scaling physical dimensions in the 3D model using a pre-surgical scan for the patient.

10. The system of claim 1, wherein the computer-implemented method further comprises: adjusting the template based on one or more structures from pre-surgical scan for the patient.

11. The system of claim 10, wherein adjusting the template comprises adjusting based on a physical dimension from a corresponding reference anatomical structure from a matched pre-surgical scan for the patient.

12. The system of claim 10, wherein adjusting the template comprises one or more of: scaling, referencing, labeling, or measuring.

13-14. (canceled)

15. The system of claim 1, wherein the computer-implemented method further comprises: importing the surgical procedure template.

16. A system for assisting in a surgical procedure, the system comprising:
one or more processors;
a memory coupled to the one or more processors, the memory storing computer-program instructions, that, when executed by the one or more processors, perform a computer-implemented method comprising:
receiving a real-time video feed of a surgical field of view;
matching a field of view specified in a surgical procedure template to the surgical field of view to generate a 3D model of the surgical field of view;
activating the surgical procedure template after a user confirms that the field of view of the surgical procedure template matches the surgical field of view;
transferring overlays from the activated template to the 3D model, wherein the overlays comprise one or more of: offsets, measurements, or zones to avoid;
displaying the 3D model with the overlays to the user;
updating, in real time, the displayed 3D model with the overlays as the surgical field of view changes from the received real-time video feed;
extracting, in real time, procedure data from the real-time video feed, wherein the procedure data comprises the surgical field of view or a modified version of the field of view;
identifying, by the one or more processors in real time, a mismatch between the predefined procedural landmark from the surgical procedure template and the extracted procedure data;
displaying visual confirmation on the display that user has not matched the predefined procedural landmark.

17. A non-transitory computer-readable storage medium comprising instructions that, when executed by one or more processors of a device, cause the device to perform operations comprising:
receiving a real-time video feed of a surgical field of view;
matching a field of view specified in a surgical procedure template to the surgical field of view to generate a 3D model of the surgical field of view;
activating the surgical procedure template after a user confirms that the field of view of the surgical procedure template matches the surgical field of view;
transferring overlays from the activated template to the 3D model, wherein the overlays comprise one or more of: offsets, measurements, or zones to avoid;
displaying the 3D model with the overlays to the user;
updating, in real time, the displayed 3D model with the overlays as the surgical field of view changes from the received real-time video feed;
extracting procedure data from the real-time video feed in real time;
identifying, by the one or more processors in real time, a match between a predefined procedural landmark from the surgical procedure template and the extracted procedure data; and
displaying visual confirmation on the display that user has matched the predefined procedural landmark.

18. The non-transitory computer-readable storage medium of claim 17, further comprising matching one or more pre-surgical patient scans to the 3D model.

19. The non-transitory computer-readable storage medium of claim 18, wherein the one or more pre-surgical patient scan comprises MRI scans.

20. The non-transitory computer-readable storage medium of claim 17, wherein updating the displayed 3D model with the overlays as the surgical field of view changes from the received real-time video feed further comprises updating the overlays.

21. The non-transitory computer-readable storage medium of claim 17, wherein the procedure data comprises the surgical field of view or a modified version of the field of view.

22. The non-transitory computer-readable storage medium of claim 17, wherein the procedure data comprises one or more of: visual data of implant position, surgical tool position, or anatomy orientation.

23-90. (canceled)
